(19) United States
(12) Patent Application Publication
(10) Pub. No.: US 2018/0011074 A1
(43) Pub. Date: Jan. 11, 2018

(54) INSTALLATION AND PROCESS OF FOLLOW-UP OF THE EVOLUTION OF THE BASICITY OF A LUBRICANT

(71) Applicant: TOTAL MARKETING SERVICES, PUTEAUX (FR)

(72) Inventors: Jean Philippe ROMAN, SEPTEME (FR); Arnaud AMIOT, RUEIL MALMAISON (FR); François CHAUDOREILLE, GRESY SUR AIX (FR); Mustapha ADJALI, AIX LES BAINS (FR); Raphael JUSTON, CHAMBERY (FR)

(73) Assignee: TOTAL MARKETING SERVICES, PUTEAUX (FR)

(21) Appl. No.: 15/549,080

(22) PCT Filed: Feb. 5, 2016
(86) PCT No.: PCT/EP2016/052451

§ 371 Date: Aug. 4, 2017

(30) Foreign Application Priority Data
Feb. 6, 2015 (FR) 1550956

(51) Int. Cl. G01N 33/28; G01N 33/30

(52) U.S. Cl. CPC G01N 33/2876; G01N 33/30

(57) ABSTRACT

This installation (2) for following up the evolution of the basicity of a lubricant circulating in a piece of equipment (M) comprises at least one conduit (4) for circulating (F1) the lubricant, this conduit being connected, upstream, to the piece of equipment (M) and the, downstream, to a recovery pan (6) as well as at least one sensor (48) for determining the basicity index of the lubricant. The installation further comprises a first controlled valve (20) for interrupting the circulation (F1) of the lubricant in the conduit (4), a buffer tank (26) for accumulating the lubricant, a first bypass line (28) connected to the conduit (4), upstream from the first valve (20) on the one hand and to the buffer tank (26) on the other hand. The installation also comprises a second controlled valve (32) for interrupting the circulation of the lubricant in the first bypass line (28), a second line (42) for discharging the lubricant, from the buffer tank (26) to the recovery pan (6) and a third controlled valve (44) for interrupting the circulation of the lubricant in the second discharge line (42). The sensor (48) is positioned on the second discharge line (42) and allows determination of the basicity index of the lubricant at the outlet of the buffer tank (26).

### CROSS-REFERENCE TO RELATED APPLICATIONS

The present application is a United States national stage application under 35 U.S.C. §371 of international patent application number PCT/EP2016/052451, filed Feb. 5, 2016, which claims priority to French patent application no. 1550956, filed Feb. 6, 2015, the entireties of which are incorporated herein by reference.

The present invention relates to an installation for following up the evolution of the basicity of a lubricant circulating in a piece of equipment, such as a ship engine. The invention also relates to a method for following up the evolution of the basicity of a lubricant.

In the field of internal combustion engines used on merchant vessels, it is known that the situation of an engine should be monitored by analyzing a lubricant circulating in this engine. Such an analysis gives the possibility of detecting wear or corrosion phenomena which tend to occur in an engine. In the past, the operation of the engines was relatively stabilized and it was sufficient to control the quality of a lubricant in an one-off way, during calls for anticipating the maintenance operations to be carried out. Nowadays, the engines are increasingly elaborated and sensitive to wear and corrosion phenomena, so that analyses have to be conducted at sea, notably for tracking the Base Number or BN of the engine oil. This imposes training of the personnel and loading on board elaborated equipment, the operation of which is relatively difficult to control, even by a trained seaman. Further, this increases the workload of the personnel.

Within this framework, providing a system for analyzing the TBN (Total Base Number) is known from the article [symbol] A low cost mid-infrared sensor for on line contamination monitoring of lubricating oils in marine engines [symbol] of Ben Mohammadi et al. (Optical Sensing and Detection Conference—Brussels—12-15.4.2010), which corresponds to the total basicity number, by means of a sensor in which a sample of the lubricant to be investigated is positioned. The piece of equipment used is elaborated and its handling is complex. If such a piece of equipment were to be loaded onboard a ship, its application would require regular sampling on the engine of the ship of an amount of oil intended to form a measurement sample. This would be both long and complex.

WO-A-03/073075 discloses a method for analyzing the basicity of a lubricant during which a measurement, carried out on a sample of a lubricant to be controlled, is compared with measurements carried out on samples of a reference lubricant. There again, this approach is provided for operation in a laboratory and requires a qualified workforce.

WO-A-2010/046591 provides the use of a system loaded on board in which the oil flowing out from an engine is directed towards a functional component associated with a measurement system giving the possibility of determining its basicity number. In practice, the oil flow rate leaving the engine is low and the flow at the outlet of the engine consists of droplets which trickle inside a duct, to the point that it is not certain that the functional component be supplied with sufficient oil flow rate so that the measurements which it carries out are correct.

US-A-2007/0084271 teaches determination of the basicity index of a lubricant by means of a signal generator coupled with a current sensor. Considering the equipment used, this approach is complex to apply and sensitive to perturbations.

These problems are not only posed on two-stroke or four-stroke propulsion engines of ships but also on other secondary engines also loaded onboard ships, for example for accessories of the hoist type. Such problems are also posed for gearboxes for equipment onboard ships or of fixed installations, such as a tidal turbine or a wind turbine. Generally, the monitoring of basicity of a lubricant is important for all lubricated equipment and known techniques are not very suitable for automation.

These are the drawbacks which the invention more particularly means to remedy by proposing a novel installation for following up the evolution of the basicity of a lubricant circulating in a piece of equipment which is adapted for operating in a simple and autonomous way, which notably discharges the onboard personnel of a ship from repeated and elaborated tasks.

For this purpose, the invention relates to an installation for following up the evolution of the basicity of a lubricant circulating in a piece of equipment, this installation comprising at least one conduit for circulating the lubricant, this conduit being connected, upstream to the relevant piece of equipment and, downstream to a pan for recovering the lubricant, as well as at least the one sensor for determining in the basicity index of the lubricant. According to the invention, this installation further comprises a first controlled valve for interrupting the circulation of a lubricant in the conduit, and a buffer tank for accumulating lubricant. According to the invention, the installation comprises a first bypass line, a second line for discharging the lubricant, from the buffer tank to the recovery pan, this second line being positioned downstream from the first bypass line and a third controlled valve for interrupting the circulation of the lubricant in the second discharge line. Further the sensor is positioned on the second discharge line and allows determination of the lazy city index of the lubricant at the outlet of the buffer tank.

By means of the invention, the buffer tank is used for accumulating an amount of lubricant sufficient for allowing proper supply of the basicity index sensor.

The lubricant which is relevant in the present invention comprises at least one lubricating base oil. Generally, the lubricating base oils may be oils of mineral, synthetic or vegetable origin as well as mixtures thereof. The mineral or synthetic oils generally used belong to one of the groups I to V according to the classes defined in the API classification (or their equivalents according to the ATIEL classification) as summarized below. The API classification is defined in American Petroleum Institute 1509 “Engine oil Licensing and Certification System” 17th edition, September 2012. The ATIEL classification is defined in “The ATIEL Code of Practice”, number 18, November 2012.

| | Content of | | |
|---|---|---|---|
| | saturated | Sulfur | |
| | substances | content | Viscosity index |
| Group I mineral oils | <90% | >0.03% | 80 ≦ VI < 120 |
| Group II hydrocracked oils | ≧90% | ≦0.03% | 80 ≦ VI < 120 |
| Group III | ≧90% | ≦0.03% | ≧120 |
| hydrocracked or | | | |
| hydro-isomerized oils | | | |

| | |
|---|---|
| Group IV | PAO (Poly alpha olefins) |
| Group V | Esters and other bases not included in the |
| | bases of group I to IV |

The mineral oils of Group I may be obtained by distillation of selected naphthenic or paraffinic crude oils and then by purification of the distillates obtained by methods such as extraction with a solvent, de-waxing with a solvent or catalytic de-waxing, hydrotreatment or hydrogenation. The oils of Groups II and III are obtained by more elaborate purification methods, for example by a combination of treatment selected from hydrotreatment, hydrocracking, hydrogenation and catalytic de-waxing. Examples of synthetic base oils of Group IV and V include polyisobutenes, alkylbenzenes and poly-alpha-olefins such as polybutenes or further esters.

In lubricants, the lubricating base oils may be used alone or as a mixture. For example, mineral oil may be combined with synthetic oil.

The cylinder oils for two-stroke marine engines are generally characterized by a viscosimetric grade SAE-40 to SAE-60, generally SAE-50 equivalent to a kinematic viscosity at 100° C. comprised between 16.3 and 21.9 mm²/s measured according to the ASTM D445 standard. The oils of grade SAE-40 have a kinematic viscosity of 100° C. comprised between 12.5 and 16.3 cSt measured according to the ASTM D445 standard. The oils of grade SAE-50 have a kinematic viscosity at 100° C. comprised between 16.3 and 21.9 cSt measured according to the ASTM D445 standard. The oils of grade SAE-60 have a kinematic viscosity at 100° C. comprised between 21.9 and 26.1 cSt measured according to the ASTM D445 standard. The lubricants used with the invention preferably have a kinematic viscosity measured according to the ASTM D445 standard at 100° C. ranging from 12.5 to 26.1 cSt, preferentially from 16.3 to 21.9 cSt. In order to obtain such a viscosity, these lubricants may further comprise one or several additives. Typically, a conventional formulation of a lubricant for marine engines, for example two-stroke engines, is of grade SAE-40 to SAE-60, preferentially SAE-50 (according to the SAE J300 classification) and comprises at least 40% by weight of a lubricant base oil of mineral, synthetic origins or mixtures thereof, adapted to the use for a marine engine. For example, a lubricating base oil of group I, according to the API classification, may be used for formulating a cylinder lubricant. The lubricating base oils of group I have a Viscosity Index (VI) ranging from 80 to 120; their sulfur content is greater than 0.03% and their content of saturated hydrocarbon compounds is less than 90%.

The lubricant may further comprise an additive selected from overbased detergents or neutral detergents. The detergents are typically anionic compounds including a long lipophilic hydrocarbon chain and a hydrophilic head, the associated cation is typically a metal cation of an alkaline or earth-alkaline metal. The detergents are preferentially selected from salts of alkaline or earth-alkaline metals (notably preferentially calcium, magnesium, sodium or barium) of carboxylic acids, sulfonates, salicylates, naphthenates, as well as phenate salts. These metal salts may contain the metal in an approximately stoichiometric amount relatively to the anionic group(s) of the detergent. In this case, these are referred to as non-overbased or <<neutral>> detergents, although they also provide some basicity. These <<neutral>> detergents typically have a BN, measured according to ASTM D2896, of less than 150 mg KOH/g, or less than 100 mg KOH/g, or further less than 80 mg KOH/g of detergent. This type of so-called neutral detergents may contribute for one part to the BN of the lubricants. Neutral detergents of the type: carboxylates, sulfonates, salicylates, phenates, naphthenates of alkaline and earth alkaline metals, for example calcium, sodium, magnesium, barium, are for example used. When the metal is in excess (in an amount greater than the stoichiometric amount relatively to the anionic group(s) of the detergent), one is dealing with so-called overbased detergents. Their BN is high, greater than 150 mg KOH/g of detergent, typically ranging from 200 to 700 mg KOH/g of detergent, preferentially from 250 to 450 mg KOH/g of detergent. The excess metal providing the overbased nature to the detergent appears as metal salts insoluble in oil, for example a carbonate, hydroxide, oxalate, acetate, glutamate, preferentially carbonate. In a same overbased detergent, the metals of these insoluble salts may be the same as those of the detergents which are soluble in oil or else may be different. They are preferentially selected from calcium, magnesium, sodium or barium. The overbased detergents thus appear as micelles consisting of insoluble metal salts maintained in suspension in the lubricant by the detergents as metal salts soluble in oil. These micelles may contain one or several types of insoluble metal salts, stabilized with one or several types of detergent. The overbased detergents including a single type of metal salt soluble in the detergent will generally be designated according to the nature of the hydrophobic chain of this latter detergent. Thus, they will be said to be of the phenate, salicylate, sulfonate, naphthenate type depending on whether this detergent is a phenate, salicylate, sulfonate, or naphthenate respectively. The overbased detergents will be said to be of the mixed type if the micelles comprise several types of detergents, different from each other by the nature of their hydrophobic chain. The overbased detergent and the neutral detergent may be selected from carboxylates, sulfonates, salicylates, naphthenates, phenates, and the mixed detergents associating at least two of these types of detergents. The overbased detergent and the neutral detergent are notably compounds based on metals selected from calcium, magnesium, sodium or barium, preferentially calcium or magnesium. The overbased detergent may be overbased with insoluble metal salts selected from the group of alkaline metal and earth alkaline metal carbonates, preferentially calcium carbonate. The lubricant may comprise at least one overbased detergent and at least one neutral detergent as defined above.

As mentioned above, in an embodiment of the invention, the lubricant may have a BN determined according to the ASTM D-2896 standard of at most 50, preferably of at most 40, advantageously of at most 30 milligrams of potash per gram of lubricant, notably ranging from 10 to 30, preferably from 15 to 30, advantageously from 15 to 25 milligrams of potash per gram of lubricant. In this embodiment of the invention, the lubricant may not comprise overbased detergents based on alkaline or earth-alkaline metals with metal carbonate salts.

In another embodiment of the invention, the lubricant has a BN determined according to the ASTM D-2896 standard of at least 50, preferentially of at least 60, more preferentially of at least 70, advantageously from 70 to 100.

The lubricant may also comprise at least one additional additive selected from dispersants, anti-wear additives or any other functional additive. The dispersants are well known additives used in the formulation of a lubricant, notably for application in the marine field. Their primary role is to maintain in suspension the initially present particles or appearing in the lubricant during its use in the engine. They prevent their agglomeration by acting on the steric hindrance. They may also have a synergistic effect on neutralization. The dispersants used as additives for a lubricant typically contain a polar group, associated with a relatively long hydrocarbon chain, generally containing from 50 to 400 carbon atoms. The polar group typically contains at least one nitrogen, oxygen or phosphorus element. The compounds derived from succinic acid are dispersants which are particularly used as lubrication additives. In particular succinimides are used, obtained by condensation of succinic anhydrides and of amines, succinic esters obtained by condensation of succinic anhydrides and of alcohols or polyols. These compounds may then be treated with various compounds notably sulfur, oxygen, formaldehyde, carboxylic acids and compounds containing boron or zinc in order to produce, for example, succinimide borates or succinimides blocked with zinc. Mannich bases, obtained by polycondensation of phenols substituted with alkyl groups, of formaldehyde and of primary or secondary amines, are also compounds used as dispersants in lubricants. In an embodiment of the invention, the dispersant content may be greater than or equal to 0.1%, preferably from 0.5 to 2%, advantageously from 1 to 1.5% by weight based on the total weight of the lubricant. The anti-wear additives protect the surfaces subject to friction by forming a protective film adsorbed on these surfaces. The most currently used is zinc di-thiophosphate or DTPZn. In this category, various phosphorus-containing, sulfur-containing, nitrogen-containing, chlorine-containing and boron-containing compounds are also found. There exists a large variety of anti-wear additives, but the most used category is that of phosphorus-sulfur-containing additives such as metal alkylthiophosphates, in particular zinc alkylthiophosphates, and more specifically zinc dialkyldithiophosphates or DTPZn. The preferred compounds are of formula Zn((SP(S)(OR₁)(OR₂))₂, wherein R₁ and R₂ are alkyl groups, preferentially including from 1 to 18 carbon atoms. The DTPZn is typically present at contents of the order of 0.1 to 2% by weight based on the total weight of the lubricant. Amine phosphates, polysulfides, notably sulfur-containing olefins, are also commonly used anti-wear additives. In lubricants for marine engines are also encountered anti-wear and extreme pressure additives of the nitrogen-containing and sulfur-containing type, such as for example metal dithiocarbamates, in particular molybdenum dithiocarbamate. The esters of glycerol are also anti-wear additives. Mention may for example be made of mono-, di- and tri-oleates, monopalmitates and monomyristates. In an embodiment, the anti-wear additive content ranges from 0.01 to 6%, preferentially from 0.1 to 4% by weight based on the total weight of the lubricant.

The other functional additives may be selected from thickeners, anti-foam additives for going against the effect of the detergents, which may for example be polar polymers such as polymethylsiloxanes, polyacrylates, anti-oxidant and/or anti-rust additives, for example organo-metal detergents or thiadiazoles. The latter are known to one skilled in the art. These additives are generally present at a weight content from 0.1 to 5% based on the total weight of the lubricant.

According to advantageous but non-mandatory aspects, an installation according to the invention may incorporate one or several of the following features, taken in any technically admissible combination:

The installation comprises means for gas pressurization of the inner volume of the buffer tank,

The gas pressurization means comprise a source of compressed air and a set of valves or a pneumatic distributor for selectively having the inner volume of the buffer tank communicate with the compressed air source or with the ambient atmosphere.

The installation comprises means for detecting the lubricant level in the buffer tank.

The means for detecting the lubricant level in the tank comprise a gas pressure sensor in the inner volume of the buffer tank.

The installation further comprises a density, viscosity, humidity and temperature sensor also positioned on the second discharge line, as well as a sensor for the dissolved iron content of the lubricant present in the buffer tank.

Moreover, the invention relates to an automated method for following up the evolution of the basicity of a lubricant circulating in a piece of equipment, by means of an installation as mentioned above. This method comprises steps:
a) closing the first valve
b) opening the second valve and closing the third valve for supplying the buffer tank from an amount of lubricant accumulated in the conduit upstream from the first valve
c) opening the third valve for having the lubricant present in the buffer tank circulate through the second discharge line, in contact with the sensor for determining the basicity index of the lubricant.
d) using an output signal of this sensor for determining the basicity index of the lubricant.

Advantageously, such a method may incorporate one or several of the following features, taken in any technically admissible combination:

when the installation comprises means for gas pressurization of the contents of the buffer tank, a step e) after step b) and before step c) is provided and consisting of gas pressurizing the buffer tank, with a pressure comprised between 6 and 12 bars, preferably between 7 and 10 bars, still preferably equal to 7 bars.

Step c) is interrupted while a residual amount of lubricant remains in the buffer tank.

The method includes a step f) after step d) and consisting of unclogging a filter integrated to the first discharge line, by having the lubricant circulate from the buffer tank to the conduit.

The invention also relates to a method for following up the operation of a piece of equipment loaded on board a ship, this method comprising the determination, onboard the ship, of the basicity index of a lubricant of the relevant piece of equipment by applying an automated method as mentioned above.

The invention will be better understood and other advantages thereof will become more clearly apparent in the light of the description which follows of three embodiments of an installation according to its principle, only given as an example and made with reference to the appended drawings wherein:

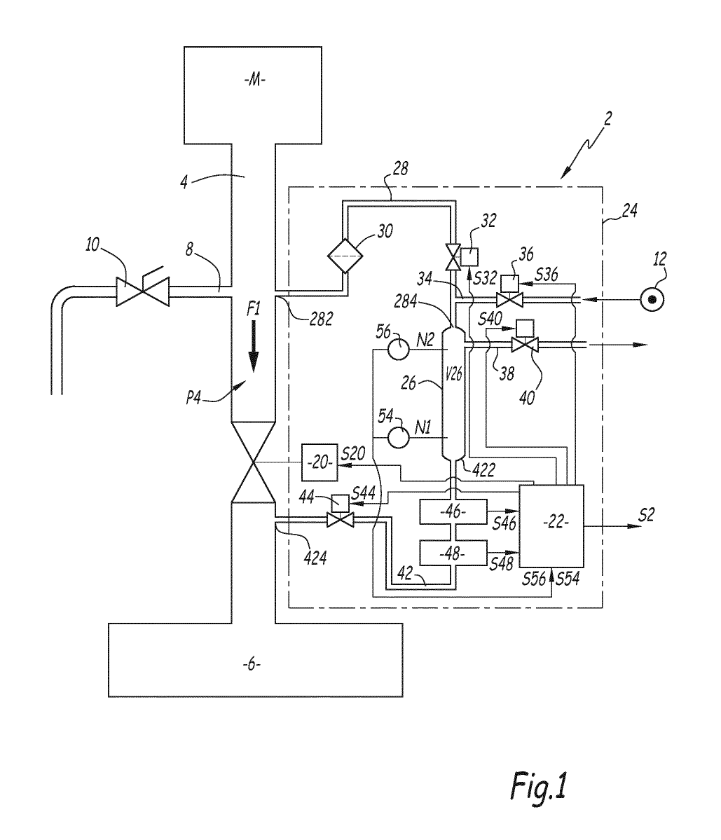

FIG. 1 is a schematic illustration of the principle of an installation according to the invention as loaded onboard a ship,

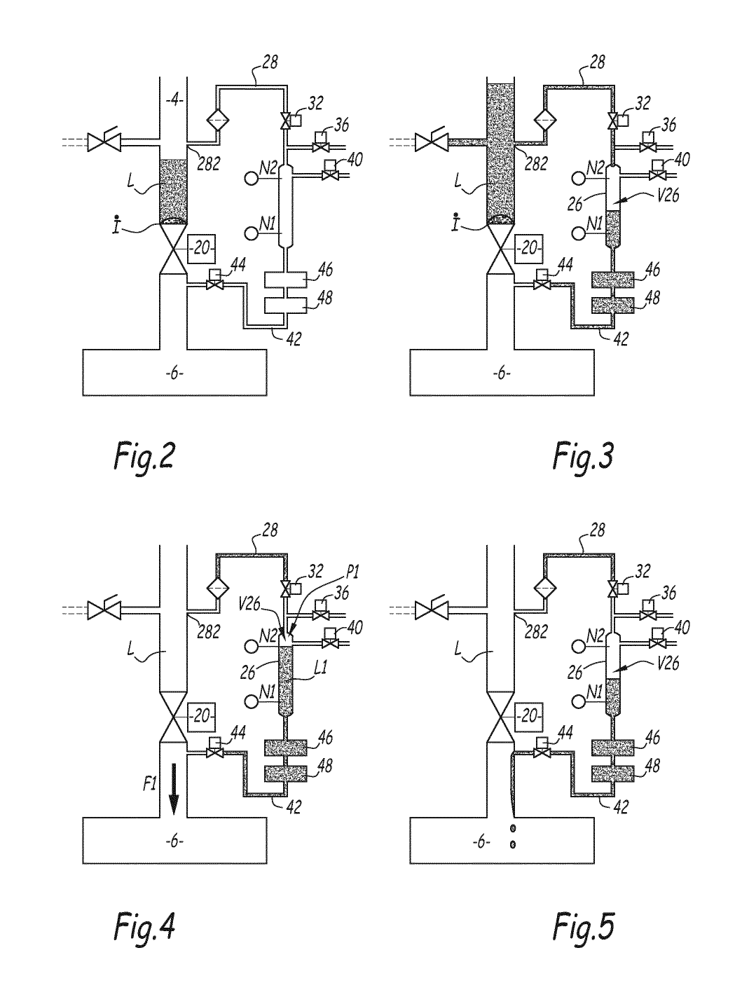

FIG. 2 is a schematic illustration at a smaller scale of the fluid portion of the installation of FIG. 1 in a first configuration of use,

FIGS. 3 to 5 are views similar to FIG. 2 when the installation is in a second, third and fourth configuration of use,

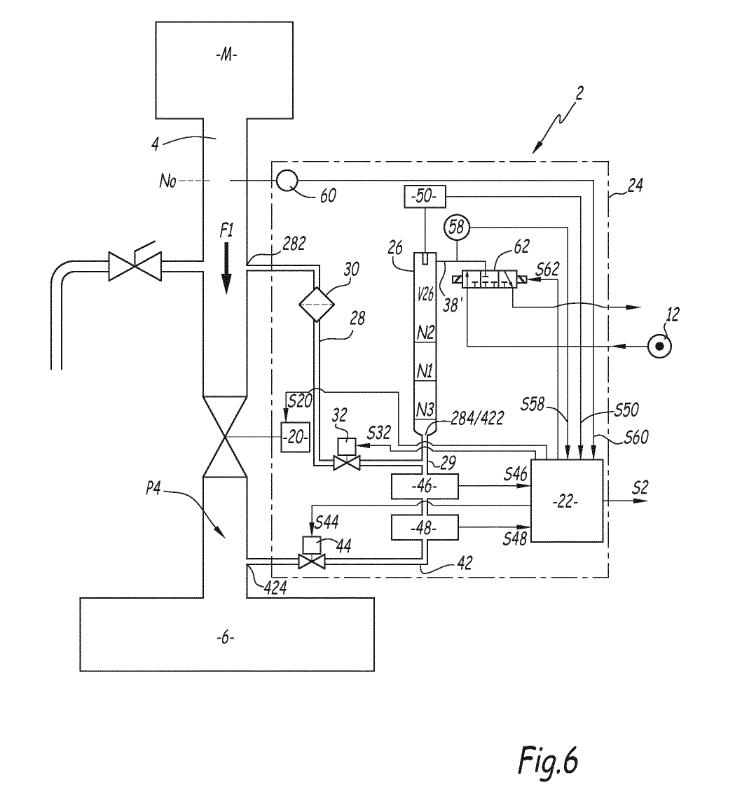

FIG. 6 is a view similar to FIG. 1 for an installation according to a second embodiment of the invention,

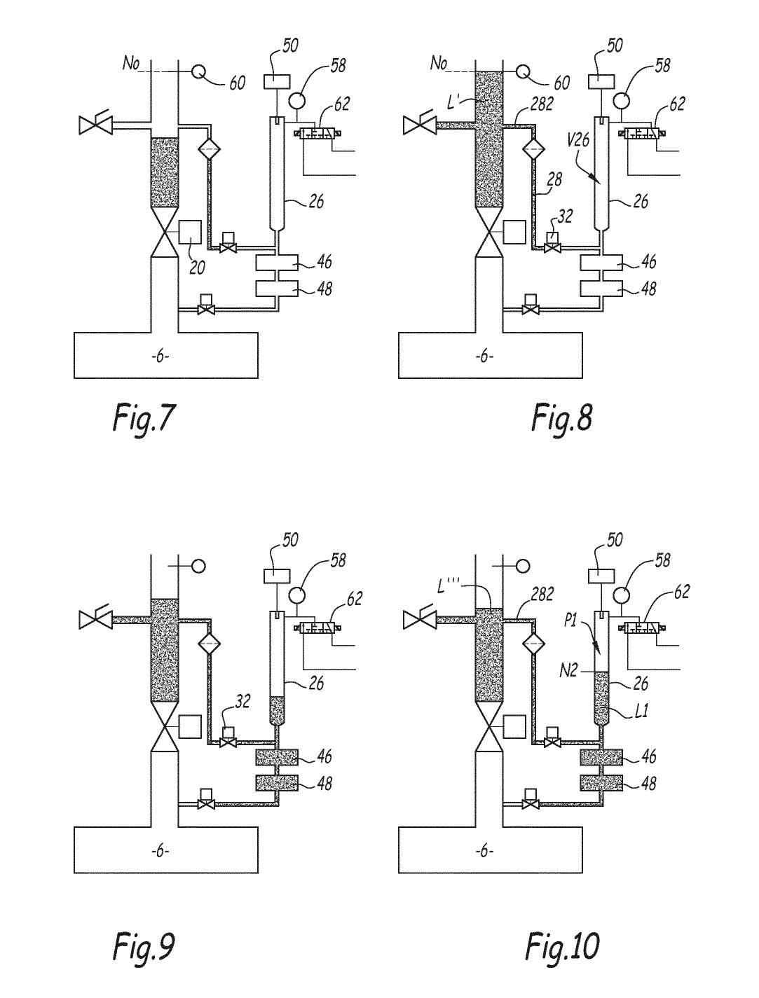

FIGS. 7 to 11 and 13 to 18 are views similar to FIG. 2 for the installation of FIG. 6 in different configurations of use,

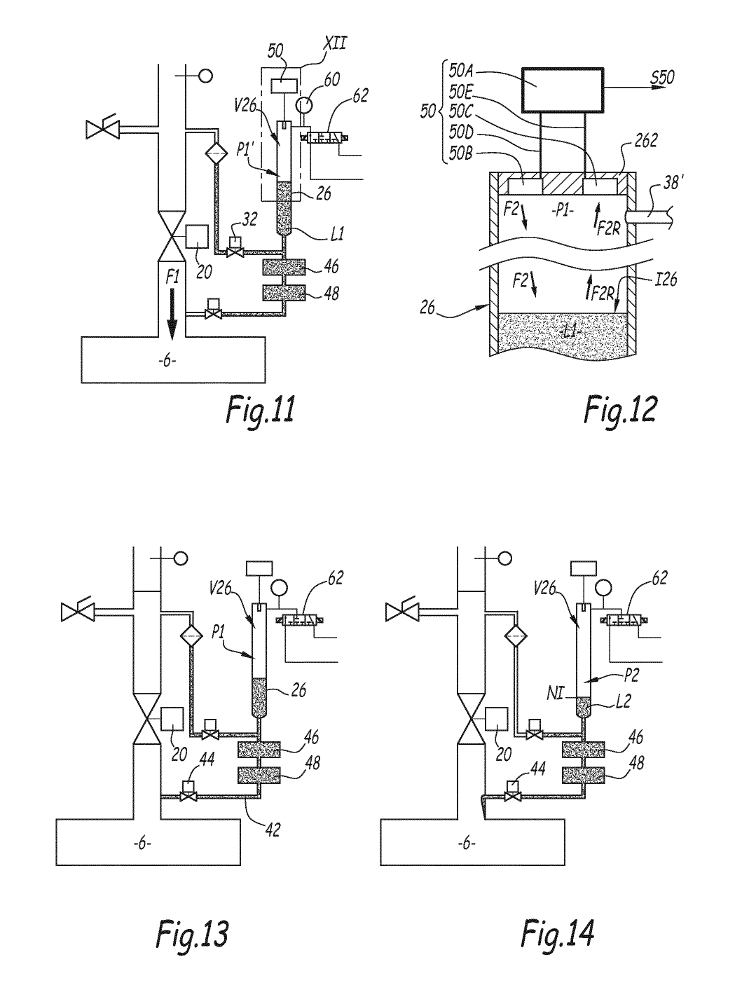

FIG. 12 is a view at a larger scale of the detail XII in FIG. 11, and

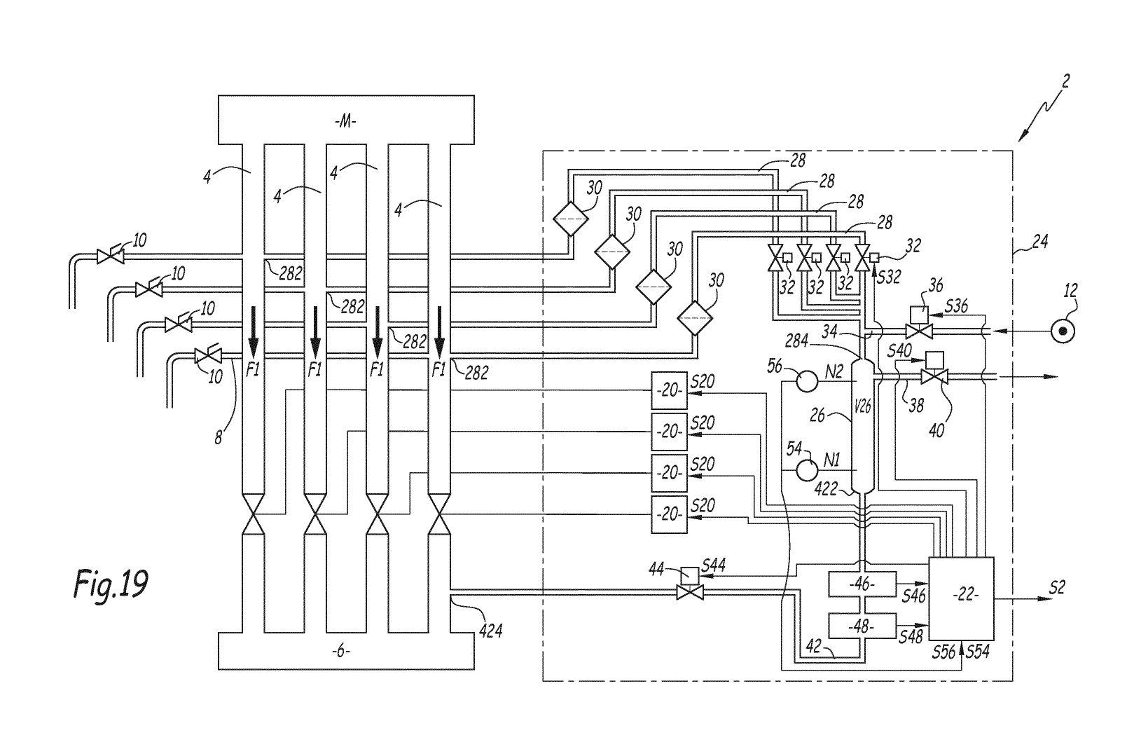

FIG. 19 is a view similar to FIG. 1 for an installation according to a third embodiment of the invention.

In FIGS. 2 to 5 and 7 to 18, the lubricant present or circulating in a portion of the installation is illustrated in gray color.

The installation 2 illustrated in FIGS. 1 to 5 is loaded onboard a ship illustrated in FIG. 1 by its engine M which includes several cylinders, for example twelve or fourteen cylinders. A conduit 4 connects the engine M to a pan 6 for recovering lubricant. In practice, the engine oil flows by gravity into the conduit 4 with a pressure P4 comprised between 1.1 and 6 bars absolute. The oil flow rate in the conduit 4 may be low, to the point that the oil trickles on the internal wall of this conduit.

This conduit 4 extends vertically, from top to bottom, from the engine M towards the pan 6. In this embodiment, the oil flowing in the conduit 4 stems from at least one cylinder of the engine M.

A capping orifice 8 is provided on the conduit 4 and equipped with a manually controlled valve 10, which gives the possibility of sampling an amount of oil flowing out of the engine M in order to proceed with physico-chemical analyses, according to an approach known per se.

The installation 2 comprises a stop valve 20 mounted on the conduit 4 and which gives the possibility of selectively interrupting the flow of oil into the conduit 4, towards the pan 6. The stop valve 20 is controlled by an electronic unit 22 by means of an electrical signal S20.

As exclusively visible in FIG. 1, the installation 2 comprises a casing 24, illustrated by its axis line mark and inside which are positioned the constitutive elements of the installation 2, except for the portion of the stop valve 20 which is integrated to the conduit 4.

The installation 2 also comprises a buffer tank 26 which is positioned in the casing 24 and which is connected to the conduit 4 by means of a first bypass line 28.

The mouth of the line 28 is noted as 282. This mouth is positioned upstream from the valve 20 on the conduit 4. The first bypass line 28 is equipped, from its mouth 282 to its opening mouth 284 in the buffer tank 26, with a filter 30, with a stop valve 32 and a tapping orifice 34. The filter 30 is used for preventing impurities with a too large size of flowing into the first bypass line 28. The stop valve 32 gives the possibility optionally of clearing or closing the first bypass line 28. The valve 32 is controlled by the electronic unit 22, by means of an electric signal S32. The tapping orifice 34 is connected, through a controlled valve 36, to a pressurized air source 12 which is not part of the installation 2 but which belongs to the standard equipment of a ship.

In practice, the pressurized air source 12 may be a compressor onboard the ship and which supplies a compressed air network which is also used at equipment other than the installation 2. Alternatively, the source 12 may be a pump dedicated to the installation 2.

The installation 2 also comprises a tapping orifice 38 connected to the tank 26, on which is mounted a stop valve 40 and which gives the possibility of putting the inner volume V26 of the tank 26 in communication with the ambient atmosphere.

In this embodiment, the tapping orifices 34 and 38 are independent. Alternatively, they may be replaced by a single tapping orifice, connected to the first line 28 or directly to the tank 26, on which the valves 36 and 40 are mounted in parallel, while being respectively connected to the pressurized air source 12 and the ambient atmosphere. In this case, it is possible to combine the valves 36 and 40 as a single three-way valve.

The valves 36 and 40 are controlled by the electronic unit 22 by means of respective electric signals S36 and S40.

The installation 2 also comprises a second line 42 for discharging the lubricant, from the inner volume V26 of the tank 26 to the recovery pan 6. The second discharge line 42 is therefore positioned downstream from the first bypass line 28 and from the tank 26, on the flow path of the lubricant. In the example, the second line 42 extends from the tank 26 to the conduit 4. Its mouth 422 is located in the low portion of the tank 26, while its outflow mouth 424 is positioned on the conduit 4, downstream from the stop valve 20, as illustrated in the figures, which gives the possibility of reducing the period of an analysis cycle since the stop valve 20 may be closed in order to generate an oil column in the conduit 4, while measuring steps take place. Alternatively, the outflow mouth 424 of the second line 42 is positioned upstream from the stop valve 20, which gives the possibility of carrying out simultaneously steps for emptying and for unblocking the filter 30 and optionally reducing the cost of the installation 2.

The second line 42 is equipped with a stop valve 44 which is controlled by the electronic unit 22 by means of an electric signal S44.

Two sensors 46 and 48 are positioned on the line 42, upstream from the valve 44.

The sensor 46 gives the possibility of measuring the density D, viscosity V, the humidity H and the temperature T of a liquid present or flowing in the second line 42. This sensor may be of the type of the one marketed by an AVENISENSE under the name of Cactus. Alternatively, the sensor 46 may be of another type or only allow measurement of a single one or some of the parameters mentioned above.

The sensor 48 is a basicity index sensor or BN, sometimes called an alkalinity index. This may be a sensor operating with infrared technology, in the medium infrared, or any other sensor adapted to the determination of the BN of a lubricant.

The installation 2 also comprises a first level sensor 54 and a second level sensor 56 which respectively give the possibility of detecting when the amount of oil in the tank 26 attains a first level N1 or a second level N2. The output electric signals S54 and S56 from the sensors 54 and 56 are delivered to the unit 22.

Alternatively, the sensors 54 and 56 may be replaced with a single sensor, such as a pressure sensor, which gives the possibility of detecting when the oil attains each of the two levels N1 and N2 in the tank 26.

FIGS. 2 to 5 schematically illustrate the successive steps of an automated method applied by means of the installation 2 of FIG. 1. This method is automated in the sense that it may be implemented, partly or preferably totally, without any human intervention, under the control of the unit 22. The same applies for the method explained hereafter concerning the second embodiment of the invention.

By default, and outside the sampling phases, the oil leaving the engine flows into the conduit 4, in the direction of the arrow F1 in FIG. 1, from the engine M to the recovery pan 6, without being retained by the valve 20 which is in an open or through-configuration, while the other valves are closed.

When the basicity index (or base number) of the oil leaving the engine M should be determined, the unit 22 drives the valve 20 to closure, so that a reservoir is generated in the conduit 4 where an oil amount accumulates, i.e. lubricant, as illustrated by the shaded portion L in FIG. 2.

In the configuration of FIG. 2, the conduit 4 is used as a decantation column and impurities I accumulate in the vicinity of the valve 20, inside the conduit 4 and in the lower portion of the lubricant amount L.

In this first step illustrated by the configuration of FIG. 2, the valves 32 and 40 are open while the valves 36 and 44 are closed.

When the lubricant level L in the conduit or column 4 attains the mouth 282, oil begins to flow through the first bypass line 28, more particularly through the filter 30 and the valve 32, as far as into the inner volume V26 of the tank 26 in which the oil flows by gravity. Indeed, the outflow mouth 284 of the first line 28 is located in the upper portion of the tank 26 and the oil may flow along the wall of the tank 26. As the valve 44 is closed, the oil gradually fills up the portion of the second discharge line 42 located upstream from the valve 44, including the internal volumes of the sensors 46 and 48, and then the inner volume V26 by driving out the air towards the atmosphere, through the valve 40. This step corresponds to the configuration illustrated in FIG. 3.

When the sensor 56 detects that the oil level N2 inside the tank 26 is attained, the unit 22 causes the installation 2 to switch to a new step, illustrated by the configuration of FIG. 4, in which the valve 20 passes into the open configuration, which allows emptying of the decantation column by directing the remainder of the lubricant amount L present upstream from the valve 20 as well as the impurities I towards the recovery pan 6. The flow in the direction of the arrow F1 therefore continues as far as into the pan 6. Moreover, the valves 32 and 40 are closed and the valve 36 is open, which gives the possibility of putting the portion of the volume V26 which is not occupied by the lubricant, i.e. the portion of this volume V26 located above the level N2, under an air pressure P1 equal to that of the air source 12, which, in the example has the value of 7 bars absolute.

This having been done, the unit 22 causes the installation 2 to switch to a next step, illustrated by the configuration of FIG. 5, when the valve 44 is open, the other valves retaining their configuration state of FIG. 4. In this case, the pressure of the air P1 in upper portion of the volume V26 has the effect of pushing the oil into the second discharge line 42, through the sensors 46 and 48, which gives the possibility to these sensors of providing the unit 22 with signals S46, respectively S48, representative of the parameters which they have detected.

If required, the signals S46 and S48 may be processed in the unit 22 in order to determine the values of the control parameters, notably by comparison with known values for reference lubricants.

The signals S46 and S48, or extrapolated signals from these signals, may be provided to the outside of the installation 2 as a conjugate signal S2, which may be used by a central processing unit for controlling the engine M.

In practice, the passage section of the basicity index sensor 48 is of about 3 mm×0.1 mm and this passage section should be able to be supplied with a sufficient flow, for a sufficient time for carrying out the measurement of the basicity index. The construction of the installation with the tank 26 gives the possibility of generating a reserve forming an oil <<buffer>>, as the amount of oil L1 contained in the tank 26 in the configuration of FIG. 4. A portion of this oil reserve L1 may be poured, either continuously or sequentially, into the second discharge line 42 so that the sensor 48 has a sufficient amount of oil to be analyzed.

From the configuration of FIG. 5, it is possible, in a subsequent step to continue emptying of the tank 26 and of the whole of the second discharge line 42 by maintaining the valve 44 open and by continuing the injection of compressed air through the valve 36.

Alternatively, it is possible to stop the emptying of the tank 26 when the oil level attains the level N1, so as to permanently retain an amount of oil L2 in the second discharge line 42, in particular in the sensors 46 and 48, for which the active portions in contact with the oil do not risk drying. If this second approach is selected, a certain amount of oil has to be used during a next measurement, for cleaning beforehand the second discharge line 42 and for not perturbing the next measurement.

In the second and third embodiments of the invention illustrated in FIG. 6 and the following, the elements similar to those of the first embodiment bear the same references. In the following, what distinguishes these embodiments from the previous one is mainly described.

In the embodiment of FIGS. 6 to 18, the first and second lines for 28 and 42 join up at a T-shaped junction 29. Thus, the outflow mouth 284 of the first bypass line 28 coincides with the mouth 422 of the second discharge line 42. The line segment located between the tank 26 and the junction 29 is common to the first and second lines 28 and 42. This line segment opens in the low portion of the tank 26, so that the oil which flows from the conduit 4 to the tank 26 directly arrives into the low portion of this tank.

Three levels N1, N2 and N3 are defined in the tank 26, the N1 and N2 levels being comparable with those of the first embodiment.

In this second embodiment, no level sensors identical with the level sensors 54 and 56 are used, but a pressure sensor 58, the output signal s58 of which is provided to the electronic control unit 22. Moreover, a level sensor 60 is mounted in the conduit 4, upstream from the valve 20, i.e. above the latter.

Further, the tapping orifices 34 and 38 and the valves 36 and 40 of the first embodiment are replaced by a single tapping orifice 38′ on which is connected the pressure sensor 58, as well as a three-way and three-orifice distributor 62 which is connected to the pressurized air source 12 on the one hand and to the ambient atmosphere on the other hand. The distributor 62 is controlled by the unit 22 by means of a dedicated electric signal S62.

The installation 2 also comprises a third sensor 50, mounted in the upper portion of the tank 26 and positioned for aiming the interface 126 between an amount of lubricant present in the tank 26 and the air present above this amount. The sensor 50 is a sensor using LIBS (Laser-Induced Breakdown Spectroscopy) technology.

More specifically, as visible in FIG. 12, the sensor 50 comprises a control unit 50A, an emitter 50B of a laser beam directed towards the interface 126, as illustrated by the arrows F2, as well as a receiver 50C capable of receiving a beam emitted in return, from the interface 126 and illustrated by the arrows F2R. The laser beam F2 emitted by the emitter 50B excites the amount L1 of lubricant and during de-excitation, an emission of a characteristic spectrum of this amount L1 occurs as a beam F2R emitted in return. The components 50B and 50C of the sensor 50 are integrated to an upper wall 262 of the tank 26 and connected to the unit 50A through two wired connections 50D and 50E.

This technology allows the sensor 50 to determine the dissolved iron content of the oil contained in the tank 26, more particularly the content of Fe²⁺ and Fe³⁺ ions. This allows determination of the degree of corrosion of the portions of the engine in contact with the oil and, consequently, initiation of preventive or corrective maintenance actions if required.

Alternatively, another type of sensor 50, also allowing determination of the dissolved iron content of the oil contained in the tank 26, may be used. In this case, this sensor may be integrated to the second line 42, notably positioned downstream from the basicity index sensor 48.

The operation of the installation 2 is the following:

By default, the valve 20 is open and the valves 32 and 44 are closed, while the distributor 62 is in the configuration illustrated in FIG. 6 where it insulates the inner volume V26 of the tank 26 from the compressed air source 12 and from the ambient atmosphere.

When it should be proceeded with the determination of the basicity index of the oil leaving the engine M, the unit 22 activates the valve 20 by means of the signal S20 in a first step, in order to bring it into a closed configuration as illustrated in FIG. 7. In this configuration, oil is present in the first bypass line 28, between the filter 30 and the valve 32, because of an unplugging operation of the filter 30, carried out earlier and which is explained hereafter.

In this configuration, the valves 32 and 44 and the distributor 62 are closed.

The level sensor 60 is positioned so that, when the oil column retained in the conduit 4 upstream from the valve 20 attains the level N0 detected by this sensor, as illustrated in FIG. 8, a predetermined amount of lubricant L′ is present above the mouth 282. For example, the predetermined amount may be equal to 100 ml. When the level sensor 60 detects that this level N0 is attained in the conduit 4, the inner volume V26 of the tank 26 is set to atmospheric pressure by actuating the distributor 62 in order to bring it into the configuration of FIG. 8.

From this configuration, the unit 22 controls the valve 32 and the distributor 62 in a following step for achieving transfer of the amount of oil L′ from the conduit 4 to the tank 26, as illustrated by the configuration of FIG. 9. In this configuration, the valve 32 is open, while the distributor 62 is closed. The transfer of oil from the conduit 4 to the buffer tank 26 is therefore accompanied by an increase in the air pressure inside the tank 26. The compression level of the air trapped in the tank may be related, after calibration, to the initial volume of air in the tank 26 and to the volume of transferred oil.

For example, for adiabatic compression and an initial volume of air in the tank 26 equal to 160 ml, the pressure in the tank 26 attains 1.7 bars absolute for 50 ml of transferred oil.

Also, by considering a tank 26 initially containing 250 ml of air, it is possible to transfer 80 ml, i.e. the amount L1 illustrated in FIG. 10, into the tank 26 before attaining in the upper portion of the latter an air pressure P1 equal to 1.7 bars absolute. This is the example considered in the following.

In this case, the level of oil N2 is attained in the tank 26 at the step illustrated by the installation 2 in the configuration of FIG. 10.

The unit 22 then automatically controls the valves and the distributor in order to attain the configuration of FIG. 11 where the tank 26 is set under pressure through the distributor 62 which connects the volume V26 to the compressed air source 12, so that the air pressure P1′ inside the tank 26 becomes equal to 7 bars. In order that this may occur, the valve 32 was switched beforehand by the unit 22 into the closed configuration, in order to avoid a return of oil from the tank 26 to the duct 4. Moreover, in this step, the valve 20 is switched by the unit 22 into an open configuration, so that the flow of oil from the engine M to the recovery pan 6 may again occur in the direction of the arrow F1. Still in this step, the sensor 50 is used for measuring the dissolved iron content, notably the Fe²⁺ and Fe³⁺ ion content, of the amount L1 of oil present in the tank 26.

To do this, the sensor 50 aims the oil/air interface 126 which is located at the level N2 in the tank 26. The output signal S50 of the sensor 50, or a signal extrapolated from this signal, is integrated to the output signal S2 of the installation 2.

From this configuration, the unit 22 controls the distributor 62 and the valve 44 by means of the signals S62 and S44, for closing the distributor 62 and opening the valve 44 respectively and thereby attaining the configuration of FIG. 13 wherein the oil content in the tank 26 is gradually driven out of the latter because of the pressure P1 prevailing in the upper portion of the inner volume V26.

The oil therefore flows through the sensors 46 and 48 which are capable of detecting the parameters for which they are provided and of providing corresponding signals S46 and S48 to the unit 22, like in the first embodiment.

The discharge of the oil contained in the tank 26 through the second discharge line 42 may take place in several cycles, by successive expansions of the air volume trapped in the tank and successive connections to the air source 12. For a tank of 250 ml initially containing 80 ml of oil, it is possible for example to carry out three successive expansions, between 7 bars and 6.2 bars, preceded with three connections to the air source 12. This allows delivery of a total volume of 50 ml in the second discharge line 42 and attaining the configuration of FIG. 14 wherein a residual amount L2 of 30 ml of lubricant, remains in the tank 26 while being subject to a pressure P2 equal to 6.2 bars.

The three successive expansions take place by filling up beforehand and successively the tank 26 with air at 7 bars, by means of a suitable command of the distributor 62.

These three expansions give the possibility of circulating 50 ml of lubricant in the sensors 46 and 48 in three successive steps, which allows them to generate three sets of signals S46 and S48 or a set of combined signals, intended for and provided to the unit 22, and then transmitted and/or processed like in the first embodiment.

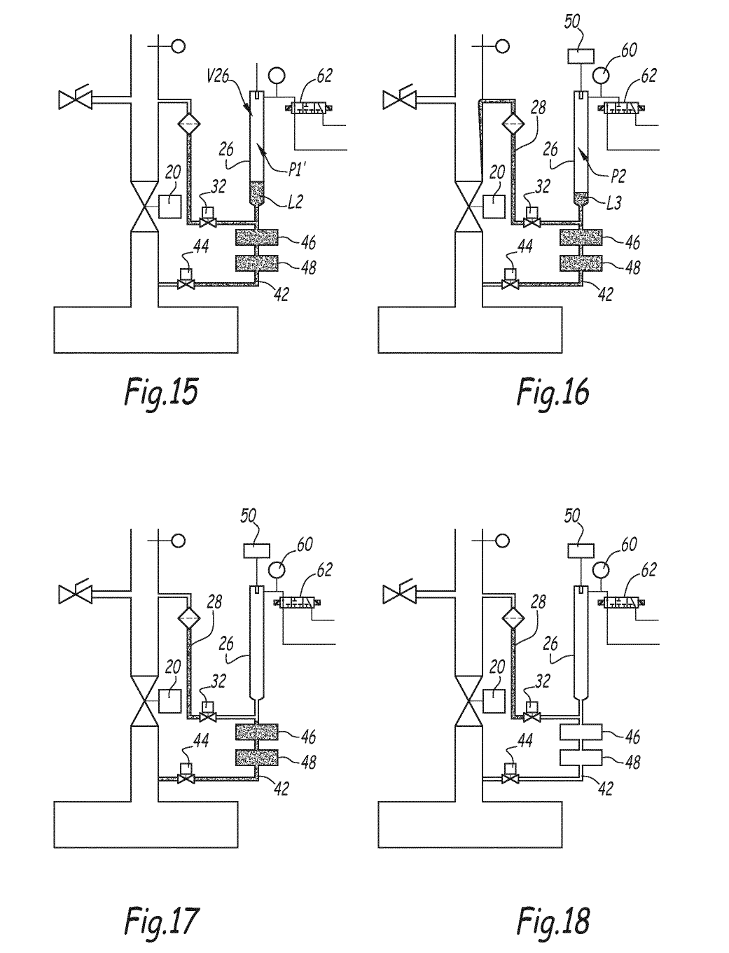

From the configuration of FIG. 14, the unit 22 causes the installation 2 to pass into the configuration of FIG. 15 wherein the inner volume V26 of the tank 26 is again pressurized to the pressure P1′ of 7 bars, in return for a suitable command of the distributor 62, while the valve 44 is closed.

Once this operation has been completed, the unit 22 orders the valve 32 to open and the distributor 62 to close, which has the effect of driving out the oil present in the lower portion of the tank 26 through the first bypass line 28, as far as into the conduit 4, in a direction for unplugging the filter 30. This step is illustrated by the configuration of FIG. 16. By lowering the pressure in the tank 26 from 7 to 6.2 bars, it is possible to have an amount of about 20 ml of the tank 26 circulate towards the conduit 4. At the end of this step, there remains an amount L3 equal to 10 ml of lubricant in the tank 26, under a pressure P2 of 6.2 bars.

Once this unclogging operation of the filter has been completed, the unit 22 causes the installation 2 to switch into the configuration of FIG. 17 wherein the valve 32 is again closed, while the valve 44 is open and the distributor 62 is placed in a configuration for supplying the volume V26 with pressurized air. This has the effect of discharging the residual amount of oil present in the second discharge line 42 and in the sensors 46 and 48 until the configuration of FIG. 18 is obtained wherein the second discharge line 42 and the sensors 46 and 48 are emptied of oil and filled with air. This corresponds to the configuration of FIG. 7 mentioned above.

It is noted in FIGS. 17 and 18 that the portion of the first bypass line 28 located between the valve 32 and the outflow mouth 284 is emptied by the air from the tank 26. This is to be related to the fact that in practice, the valve 32 is immediately positioned upstream from the junction 29.

In the third embodiment of the invention illustrated in FIG. 19, several conduits 4 are used, each of them being provided for collecting the oil from a single cylinder of the engine M.

Each conduit 4 is equipped with a valve 20 controlled by the electronic unit 22 and which gives the possibility of interrupting the flow of a stream F1 of lubricant in the relevant conduit 4. A first bypass line 28 is connected, to each conduit 4, upstream from its valve 20 on the one hand and to the inlet of the buffer tank 26 on the other hand, which is the same as in the first embodiment. The installation 2 therefore comprises as many bypass lines 28 as there are conduits 4. Starting from its mouth 282, each bypass line 28 is equipped with a filter 30 and a stop valve 32. The first four lines 28 join up downstream from their respective stop valves 32 and the tapping orifice 34 is common to the first four bypass lines 28, as well as their outflow mouth 284 in the buffer tank 26.

A tapping orifice 8 is provided on each conduit 4 and equipped with a manually controlled valve 10, according to an approach parallel to the one mentioned above concerning the first embodiment. Alternatively, only one or certain conduits 4 are equipped with such a tapping orifice 8.

The second discharge line 42 is common for all the cylinders of the engine and receives, downstream from the tank 26, the oil from all the first bypass lines 28. The outflow mouth 424 of the second discharge line is positioned on one of the conduits 4, downstream from its stop valve 20.

This third embodiment allows optimization of the size of the conduits 4 and their pathway within the engine compartment of a ship. This allows a gain in room as compared with the first embodiment.

By successively applying the method explained above concerning the first embodiment, for each of the conduits 4, the installation of this third embodiment gives the possibility of determining, by means of the sensor 48, identical with the one of the first embodiment, the basicity index of the fuel at the outlet of each of the cylinders of the engine M on which is connected a conduit 4.

In the example of FIG. 19, four conduits 4 are provided, each dedicated to a cylinder of the engine M. Alternatively, the number of conduits 4 is different, while remaining greater than or equal to 2, in order to adapt the installation 2 depending on the configuration of the engine M and on the available space for housing the conduits 4.

Regardless of the embodiment, the installation 2 allows an efficient measurement of the basicity index or BN of an oil leaving the engine M by means of a method which may be automated and which does not require any particular knowledge on behalf of a user, since the signal S2 may be directly legible, either by man or by a machine.

In practice, the maximum pressure P1′ prevailing in the inner volume V26 of the tank 26, which depends on the pressure of the source 12, is not limited to 7 bars. It is comprised between 6 and 12 bars, preferably between 7 and 10 bars depending on the pressure of the compressed air network available on the ship. The value of 7 bars is preferred since it gives good experimental results and corresponds to a currently available pressure level. It is important that this pressure P1 be greater than the pressure P4 of the oil in the conduit 4, which is comprised between 1.1 and 6 bars as mentioned above. Indeed, it is the difference between the pressures P1 and P4 which ensures the flow of the oil through the second discharge conduit 42.

Regardless of the embodiment, the installation 2, which is essentially comprised in the casing 24, is easy to install on board a ship and does not require the setting into place of the valve 20 in the conduit 4, the junction of the lines 28 and 42 on this conduit and its supply with current and pressurized air. The installation 2 may therefore be easily implanted on a new ship or used for retrofitting an operating ship.

The invention is described above in the case of its use for a ship propulsion engine. However it may be applied to other pieces of equipment, for example an auxiliary or ancillary ship engine, as well as a gearbox, notably of a tidal or wind turbine.

In the foregoing, the terms [symbol] oil [symbol] and [symbol] lubricant [symbol] are used indistinctly since an engine oil is a lubricant. The invention may however be applied to other lubricants such as oils for transmissions and for gears, oils for compressors, hydraulic oils, turbine oils or further oils for centrifuges.

The features of the embodiments and alternatives contemplated above may be combined for generating novel embodiments of the invention.

1. An installation for following up the evolution of the basicity of a lubricant circulating in a piece of equipment, this installation comprising:
at least one conduit for circulating the lubricant, this conduit being connected upstream to the piece of equipment and downstream to a recovery pan,
at least one sensor for determining the basicity index of the lubricant,
a first controlled valve for interrupting the circulation of the lubricant in the conduit,
a buffer tank for accumulating the lubricant,
wherein the installation comprises:
a first bypass line connected to the conduit, upstream from the first valve, on the one hand and to the buffer tank on the other hand,
a second controlled valve for interrupting the circulation of the lubricant in the first bypass,
a second line for discharging the lubricant, from the buffer tank to the recovery pan, this second line being positioned downstream from the first bypass line,
a third controlled valve for interrupting the circulation of the lubricant in the second discharge line
and wherein the sensor is positioned on the second discharge line and allows determination of the basicity index of the lubricant at the outlet of the buffer tank.

2. The installation according to claim 1, wherein it comprises means for gas pressurization of the inner volume of the buffer tank.

3. The installation according to claim 2, wherein it gas pressurization means comprise a source of compressed air and a set of valves or a pneumatic distributor for selectively putting the inner volume of the buffer tank in communication with a source of compressed air or the ambient atmosphere.

4. The installation according to one of the preceding claims, wherein it comprises means for detecting the lubricant level in the buffer tank.

5. The installation according to claim 4, wherein it comprises means for gas pressurization of the inner volume of the buffer tank and wherein the means for detecting the lubricant level in the tank comprise a gas pressure sensor in the inner volume of the buffer tank.

6. The installation according to claim 1, wherein it further comprises,
a sensor for measuring density, viscosity, humidity and temperature also positioned on the second discharge line,
a sensor for measuring the dissolved iron content of the lubricant present in the buffer tank.

7. An automated method for following the evolution of the basicity of a lubricant circulating in a piece of equipment, by means of an installation according to claim 1, wherein it comprises at least steps of:
a) closing the first valve,
b) opening the second valve and closing the third valve for supplying the buffer tank from an amount of lubricant accumulated in the conduit, upstream from the first valve,
c) opening the third valve for circulating the lubricant present in the buffer tank through the second discharge line, in contact with the sensor for determining the basicity index of the lubricant,
d) using an output signal of this sensor for determining the alkalinity of the lubricant.

8. The method according to claim 7, wherein it is applied with an installation comprising means for gas pressurization of the inner volume of the buffer tank and wherein it comprises a step e) after step b) and before step c) and consisting of:
e) gas pressurizing the inner volume of the buffer tank, with a pressure comprised between 6 and 12 bars, preferably between 7 and 10 bars, still preferably 7 bars.

9. The method according to claim 7, wherein step c) is interrupted, while a residual amount of lubricant remains in the buffer tank.

10. The method according to claim 9, wherein it comprises a step f) after step d) and consisting of:
f) unclogging a filter integrated to the first bypass line by having the lubricant circulate from the buffer tank to the conduit.

11. A method for following up the operation of a piece of equipment on board a ship, wherein it comprises the determination, onboard the ship, of the basicity index of a lubricant of the piece of equipment by applying a method according to claim 7.
